(19) United States
(12) Patent Application Publication
(10) Pub. No.: US 2024/0252339 A1
(43) Pub. Date: Aug. 1, 2024

(54) CERVICAL COLLAR

(71) Applicant: DeRoyal Industries, Inc., Powell, TN (US)

(72) Inventors: Walter Cleveland Cowart, Blaine, TN (US); Sarah O Davis, Knoxville, TN (US); Nicholas John Poker, Knoxville, TN (US); John Connor Brown, Knoxville, TN (US); Charles Joseph French, III, Knoxville, TN (US); Rebecca Laughon, Powell, TN (US); Dhanvin Sunil Desai, Knoxville, TN (US)

(73) Assignee: DeRoyal Industries, Inc., Powell, TN (US)

(21) Appl. No.: 18/102,889

(22) Filed: Jan. 30, 2023

(51) Int. Cl. A61F 5/055

(52) U.S. Cl. CPC A61F 5/055

(57) ABSTRACT

An article of manufacture for providing an adjustable cervical collar according to the present invention contains a lower support arm, a pivotable upper chin support arm coupled to the lower support on opposing sides, and a rear support member removably attachable from the lower support arm. The lower support arm includes a lower support arm cover, a lower support arm frame, and a pair of lower support arm locking mechanisms. The pivotable upper chin support arm is coupled to the lower support on opposing sides; the upper chin support arm includes a pair of chin support arm outer components and a chin support arm center component. The rear support member is removably attachable from the lower support arm; the rear support member includes a rear support member frame component and a pair of rear support member attachment straps for coupling the rear support member to the lower support arm.

### TECHNICAL FIELD

This application relates in general to an article of manufacture for providing a healthcare aid, and more specifically, to an article of manufacture providing an adjustable cervical collar.

### BACKGROUND

Cervical collars are useful healthcare aid devices that help a user support the neck and head while preventing the wearer from moving the head from a safe and comfortable position. The cervical collar typically is worn when the user has suffered an injury to the head, neck, and/or shoulders that will benefit from keeping the head and neck in a stationary position while the condition heals. Immobilizing a head and neck into a single position can become tiring and uncomfortable for the wearer when the cervical collar is ill-fitting and not properly supporting the head and/or neck. Adjusting a cervical collar and its supporting components to fit users of different sizes and positioning the cervical collar into a variety of configurations to support users having different injuries may be challenging.

Therefore, a need exists for an article of manufacture providing an adjustable cervical collar. The present invention attempts to address the limitations and deficiencies in prior solutions according to the principles and example embodiments disclosed herein.

### SUMMARY

In accordance with the present invention, the above and other problems are solved by providing an article of manufacture for an adjustable cervical collar according to the principles and example embodiments disclosed herein.

In one embodiment, the present invention is an article of manufacture providing an adjustable cervical collar. The adjustable cervical collar contains a lower support arm having opposing ends, a pivotable upper chin support arm coupled to the lower support arm on opposing sides, and a rear support member removably attachable from the lower support arm. The lower support arm includes a lower support arm cover, a lower support arm frame, and a pair of lower support arm locking mechanisms. The pivotable upper chin support arm includes a pair of chin support arm outer components and a chin support arm center component. The rear support member includes a rear support member frame component and a pair of rear support member attachment straps for coupling the rear support member to the lower support arm.

The foregoing has outlined rather broadly the features and technical advantages of the present invention in order that the detailed description of the invention that follows may be better understood. Additional features and advantages of the invention will be described hereinafter that form the subject of the claims of the invention.

It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features that are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

Referring now to the drawings in which like reference numbers represent corresponding parts throughout:

FIGS. 1a-c illustrate example embodiments of an article of manufacture providing an adjustable cervical collar according to the present invention.

FIGS. 2a-b illustrate various embodiments of an article of manufacture providing an adjustable cervical collar according to the present invention.

FIGS. 3a-b illustrate a rear support member coupling to a front arm assembly of an article of manufacture providing an adjustable cervical collar according to the present invention.

FIGS. 4a-e illustrate various front views of example embodiments of an article of manufacture providing an adjustable cervical collar according to the present invention.

FIGS. 5a-d illustrate various side views of coupling a lower support arm and an upper chin support arm of an article of manufacture providing an adjustable cervical collar according to the present invention.

FIGS. 6a-d illustrate various top and bottom views of an article of manufacture providing an adjustable cervical collar according to the present invention.

FIGS. 7a-d illustrate various views of coupling members between a lower support arm and an upper chin support arm of an article of manufacture providing an adjustable cervical collar according to the present invention.

FIGS. 8a-f illustrate various views of a lower support arm of an article of manufacture providing an adjustable cervical collar according to the present invention.

FIGS. 9a-h illustrate various views of a rear support member of an article of manufacture providing an adjustable cervical collar according to the present invention.

FIGS. 10a-c illustrate various views of a lower support arm with a pair of position locking mechanisms of an article of manufacture providing an adjustable cervical collar according to the present invention.

FIGS. 11a-f illustrate various views of components of each of the pair of position locking mechanisms of an article of manufacture providing an adjustable cervical collar according to the present invention.

FIGS. 12a-e illustrate open and closed configurations of each of the pair of position locking mechanisms of an article of manufacture providing an adjustable cervical collar according to the present invention.

FIGS. 13a-c illustrate multiple views of a lower support arm padding used with the lower support arm of an article of manufacture providing an adjustable cervical collar according to the present invention.

FIGS. 14a-c illustrate multiple views of a rear support member padding used with the rear support member of an article of manufacture providing an adjustable cervical collar according to the present invention,

FIGS. 15a-b illustrate multiple views of an upper chin support arm padding used with the upper chin support arm of an article of manufacture providing an adjustable cervical collar according to the present invention.

### DETAILED DESCRIPTION

This application relates in general to an article of manufacture for providing a healthcare aid, and more specifically, to an article of manufacture providing an adjustable cervical collar according to the present invention.

Various embodiments of the present invention will be described in detail with reference to the drawings, wherein like reference numerals represent like parts and assemblies throughout the several views. Reference to various embodiments does not limit the scope of the invention, which is limited only by the scope of the claims attached hereto. Additionally, any examples set forth in this specification are not intended to be limiting and merely set forth some of the many possible embodiments for the claimed invention.

In describing embodiments of the present invention, the following terminology will be used. The singular forms “a,” “an,” and “the” include plural referents unless the context clearly dictates otherwise. As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. As used herein, the singular forms “a,” “an,” and “the” are intended to include the plural forms as well, unless the context clearly indicates otherwise.

It further will be understood that the terms “comprises,” “comprising,” “includes,” and “including” specify the presence of stated features, steps, or components, but do not preclude the presence or addition of one or more other features, steps, or components. It also should be noted that in some alternative implementations, the functions and acts noted may occur out of the order noted in the figures. For example, two figures shown in succession may in fact be executed substantially concurrently or may sometimes be executed in the reverse order, depending upon the functionality and acts involved.

The terms “individual” and “user” refer to an entity, e.g., a human, using an article of manufacture providing an adjustable cervical collar according to the present invention. The term user herein refers to one or more users.

The terms “invention” or “present invention” refer to the invention being applied for via the patent application with the title “A Cervical Collar.” The invention may be used interchangeably with “the collar.”

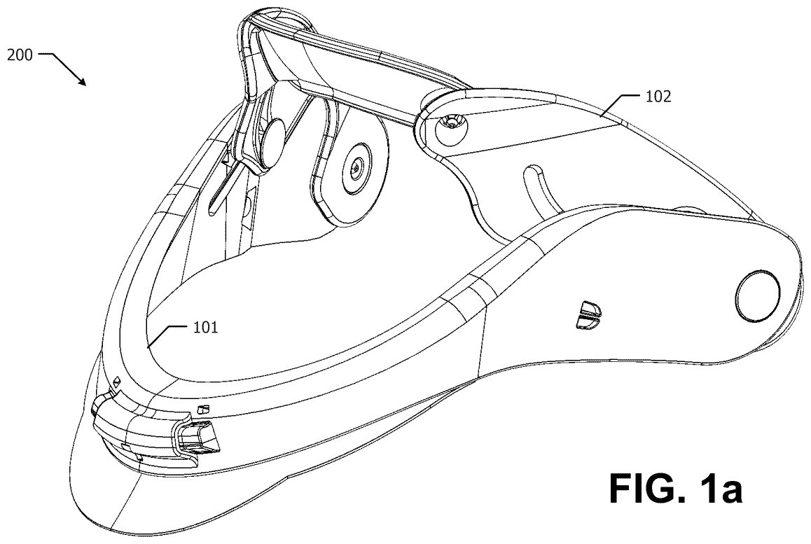

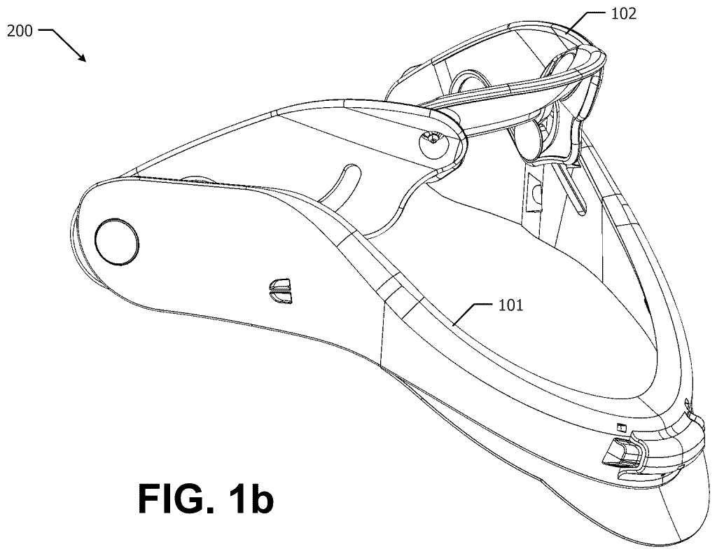

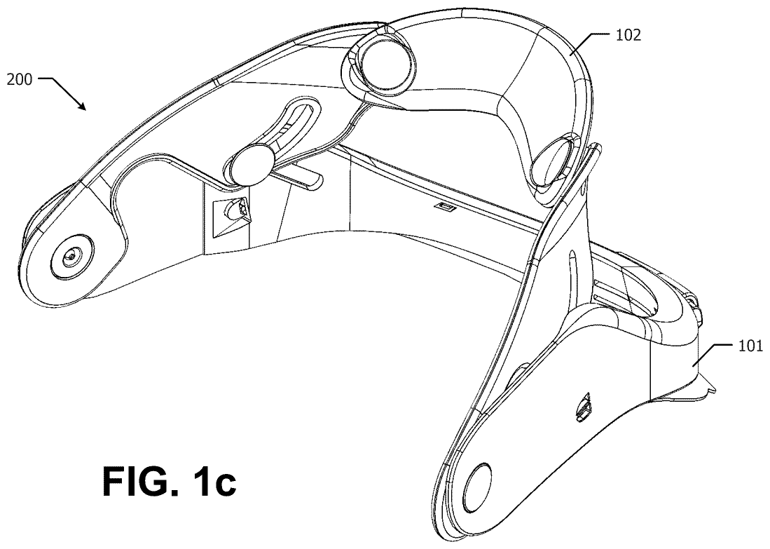

In general, the present disclosure relates to an article of manufacture providing an adjustable cervical collar according to the present invention. FIGS. 1a-c illustrate example embodiments of an article of manufacture providing an adjustable cervical collar according to the present invention. FIG. 1a shows a right perspective view of a front assembly 200 of the adjustable cervical collar 100. The front part of the cervical collar 100 includes a lower support arm 101 coupled to a pivotable upper chin support arm 102. Both lower support arm 101 and upper chin support arm 102 are U-shaped arms that are coupled together at the outer ends of the arms. The upper chin support arm 102 is positioned above the lower support arm 101 when worn by a user.

The lower support arm 101 is worn in a downward configuration in which the lower support arm 101 rests against the user's chest with the pivot point of the two arms being positioned above a user's shoulders. The upper chin support arm 102 typically extends outward to position a top portion of the upper chin support arm 102 just below the user's chin and jaw. The upper chin support arm 102 and lower support arm 101 are lockable to configure the position of the lower support arm 101 relative to the upper chin support arm 102 to remain in a user-defined configuration. With the lower support arm 101 resting upon the user's chest, the user may rest his or her chin on top of the upper chin support arm 102, allowing the front assembly 200 to support the weight of the user's head.

FIG. 1b shows a left perspective view of the adjustable cervical collar. The cervical collar 100 is symmetrical with each side of the lower support arm 101 and upper chin support arm 102 being identical. At the center of the lower support arm 101, a pair of locking mechanism release buttons 504a-b are positioned to be easily accessible to the user. When the user depresses the locking mechanism release buttons 504a-b, a locking mechanism on each side of the lower support arm 101 unlocks to permit the adjustment of the position of the lower support arm 101 relative to the upper chin support arm 102. When the lower support arm 101 and upper chin support arm 102 are in a desired configuration, the user releases the locking mechanism release buttons 504a-b to relock the locking mechanism to restrain the configuration of the lower support arm 101 and upper chin support arm 102.

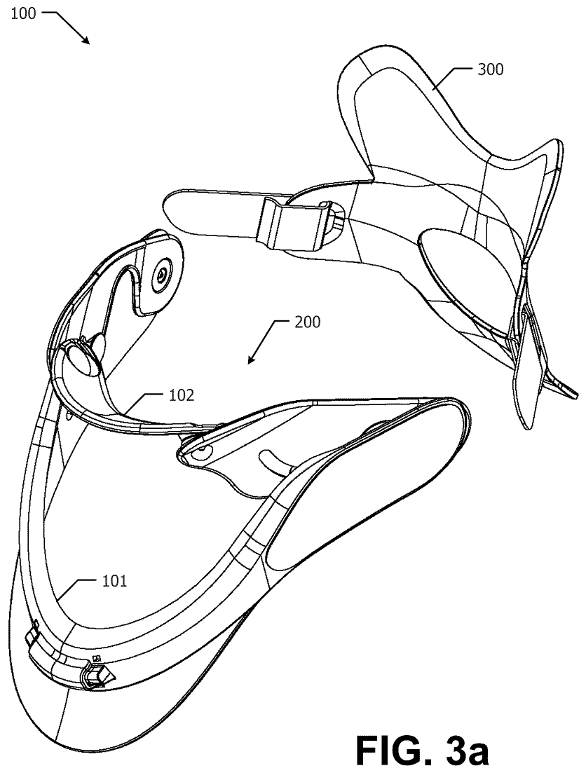

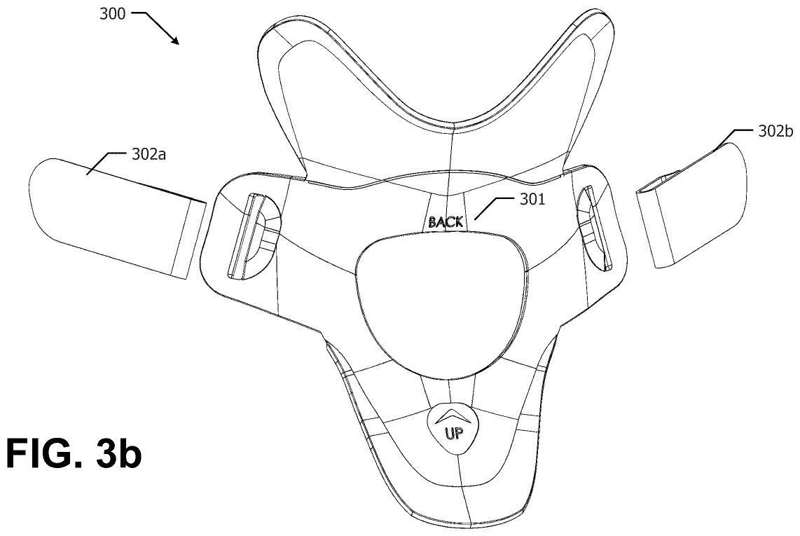

FIG. 1c shows a rear perspective view of the adjustable cervical collar. The rear side of the cervical collar 100 shows the inside surfaces of the lower support arm 101 and upper chin support arm 102 that are placed about the user's head and neck to place the cervical collar 100 in position. The lower support arm 101 and upper chin support arm 102 form a front assembly 200. The cervical collar 100 is secured to the user by attaching a rear support member 300 shown in FIGS. 3a-b to the front assembly 200. The rear support member 300 is tightened to the front assembly 200 using straps to secure the cervical collar 100 in place around the user's neck.

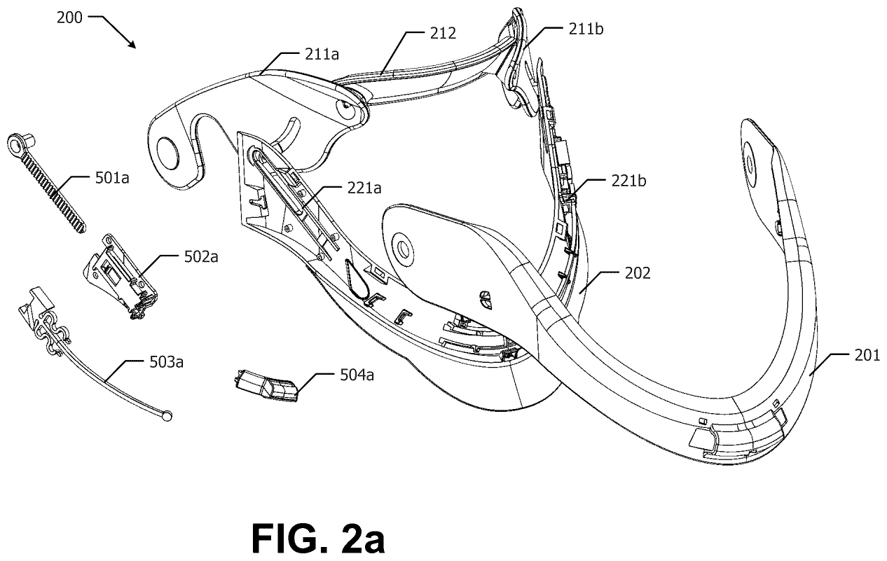

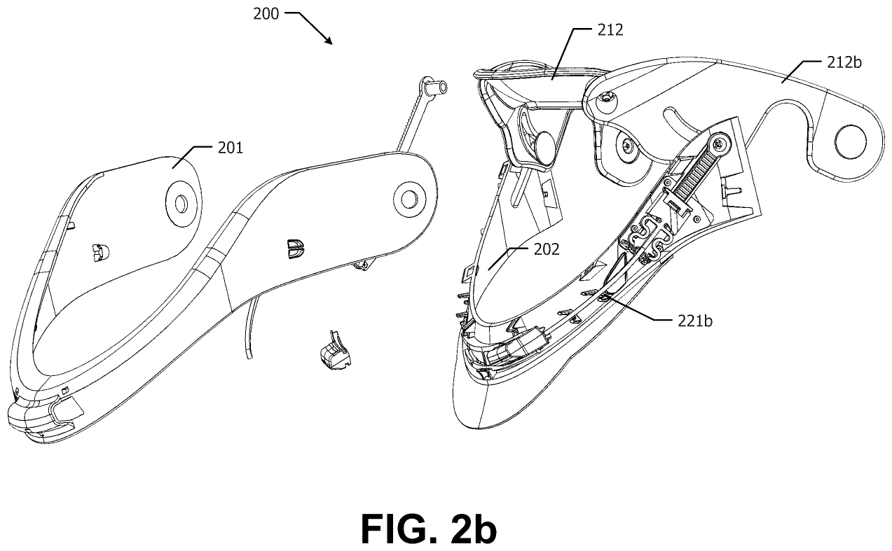

FIGS. 2a-b illustrate various embodiments of an article of manufacture providing an adjustable cervical collar according to the present invention. The lower support arm 101 is shown having a lower support arm outer shell 201 coupled over a lower support arm inner shell 202. A pair of lower support arm locking mechanisms 221a-b is coupled to both sides of the lower support arm inner shell 202 to lock and unlock the lower support arm 101 and upper chin support arm 102 in a desired configuration. The lower support arm outer shell 201 encases the lower support arm locking mechanisms 221a-b and the lower support arm inner shell 202 within the lower support arm 101. A lower end of each of the lower support arm locking mechanisms 221a-b connects each of the locking mechanism release buttons 504a-b to the pivot point between the lower support arm 101 and the upper chin support arm 102 to lock and unlock the lower support arm locking mechanisms 221a-b.

The locking mechanism release buttons 504a-b extend out of the lower support arm outer shell 201 adjacent to each other allowing the user to depress both of the locking mechanism release buttons 504a-b at the same time permitting the user to simultaneously pivot the lower support arm 101 from the upper chin support arm 102 on both sides thereby making the reconfiguration of the cervical collar 100 straightforward.

FIG. 2b shows an exploded view of the left side of the lower support arm. The alternate side of the lower support arm 101 is identical to the corresponding side of the lower support arm 101 having a similar lower support arm locking mechanism 221b under the lower support arm outer shell 201.

FIGS. 3a-b illustrate a rear support member coupling to a front arm assembly of an article of manufacture providing an adjustable cervical collar according to the present invention. FIG. 3a shows the cervical collar 100. The cervical collar 100 is shown in a configuration allowing the rear support member 300 to be coupled to the front assembly 200 by a pair of rear support member attachment loop straps 302a-b. The rear support member 300 is connected to lower support arm 101 with loop straps and adhesive hook.

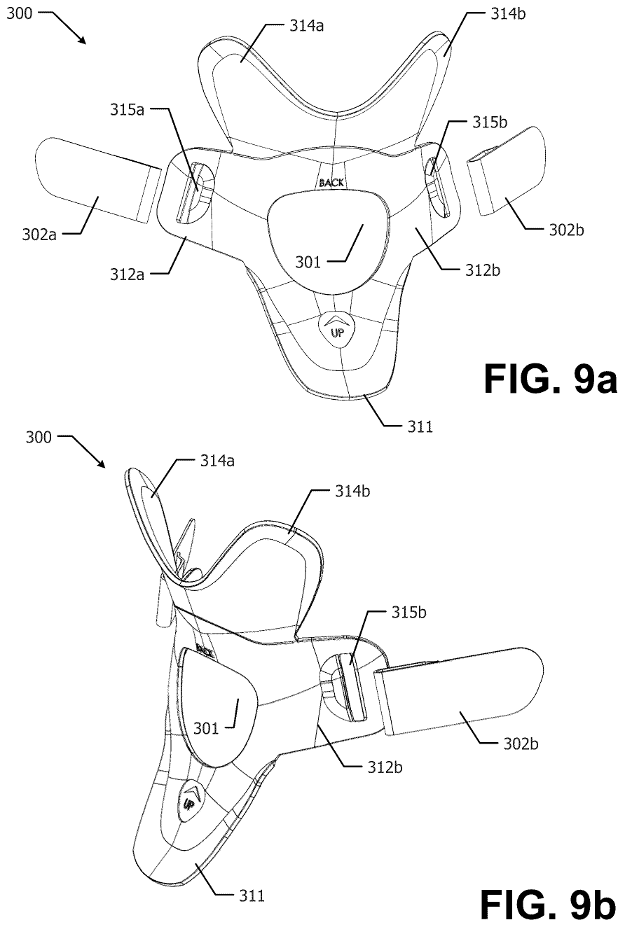

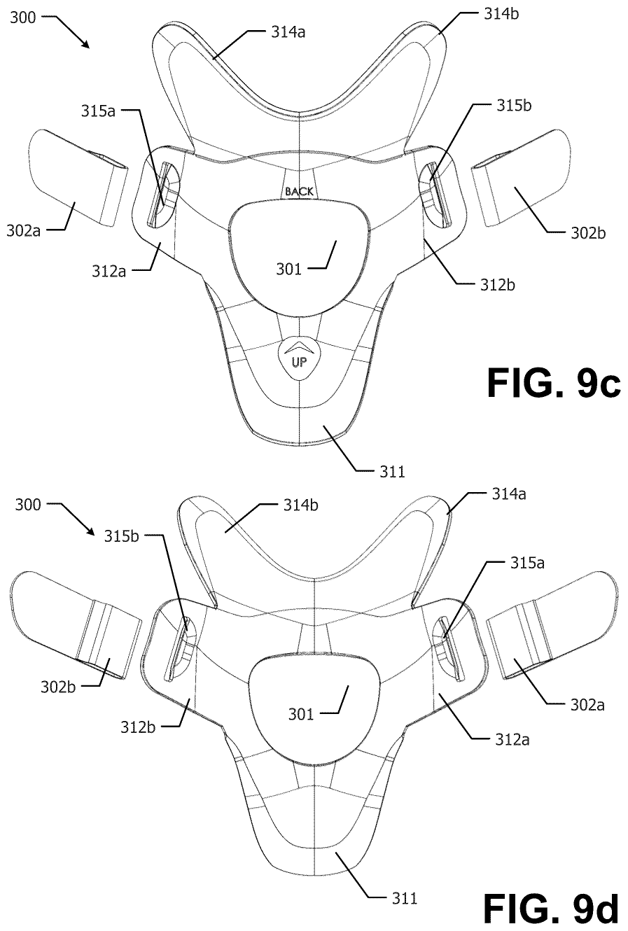

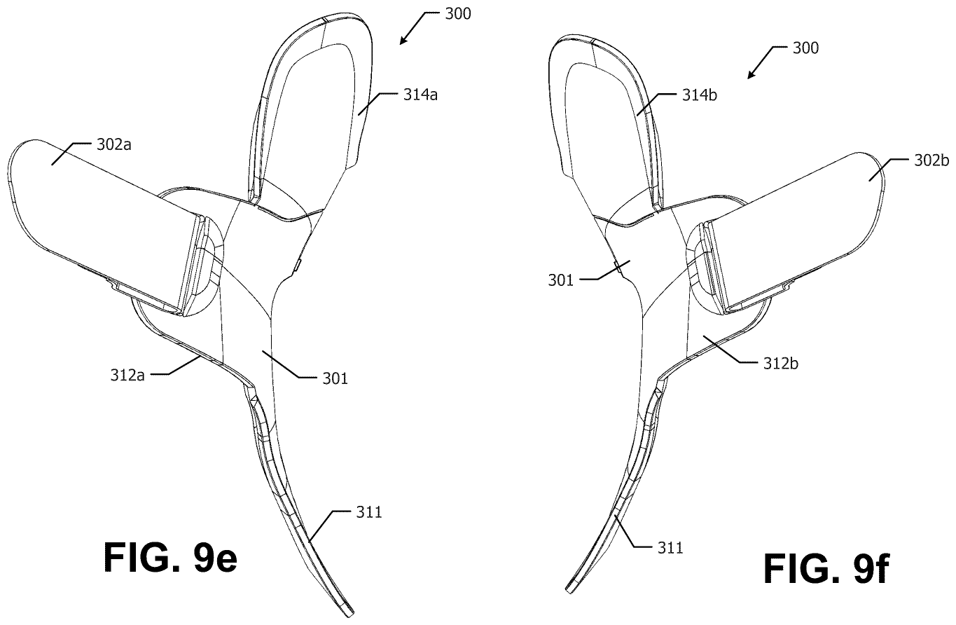

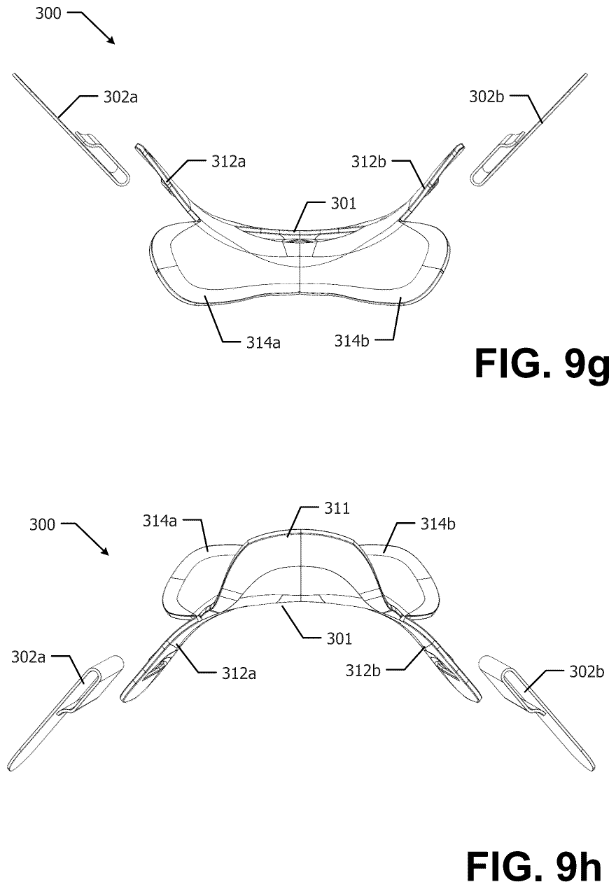

FIG. 3b shows an exploded view of the rear support member. The rear support member 300 includes a rear support member frame component 301 and rear support member attachment loop straps 302a-b. The rear support member 300 and its rear support member frame component 301 and rear support member attachment loop straps 302a-b are described in more detail below in reference to FIGS. 9a-f.

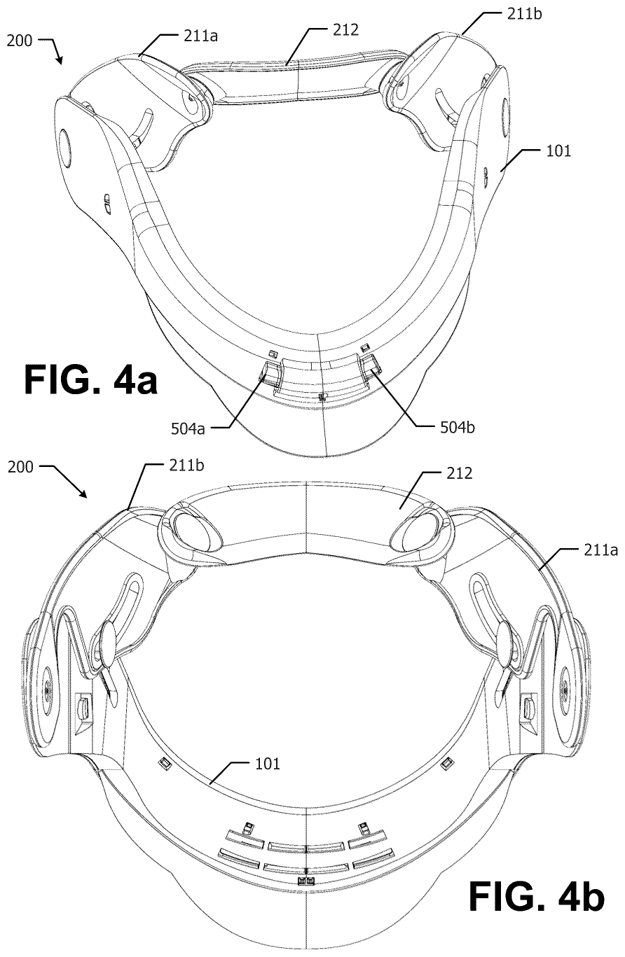

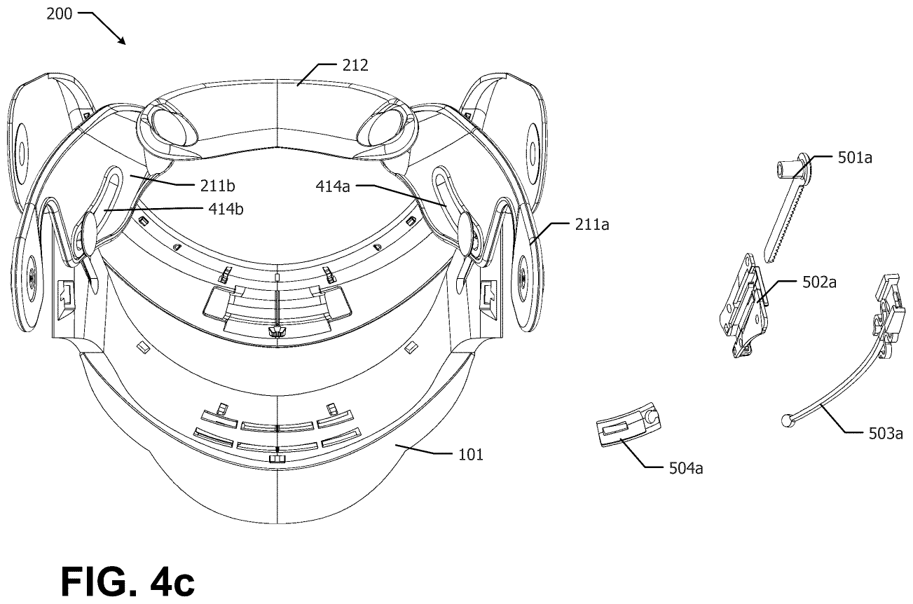

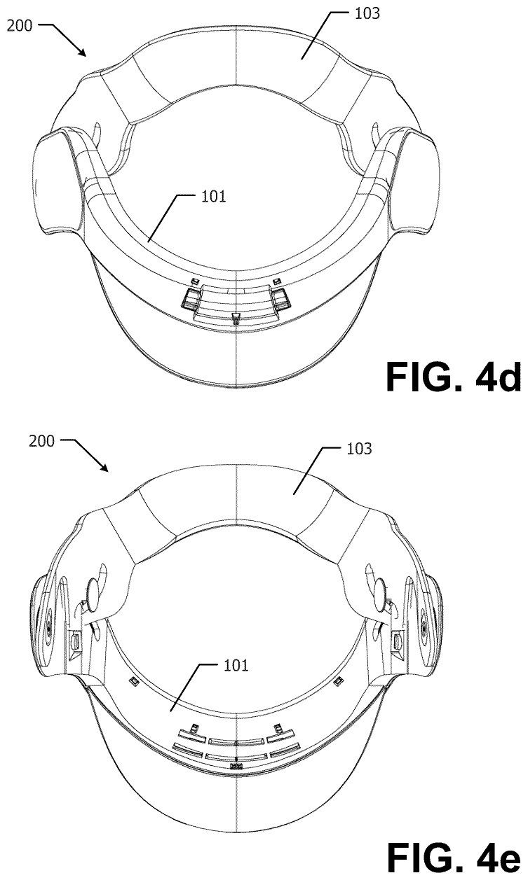

FIGS. 4a-e illustrate various views of example embodiments of an article of manufacture providing an adjustable cervical collar according to the present invention. FIG. 4a shows a front view of a front assembly 200 including the lower support arm 101 and upper chin support arm 102. The upper chin support arm 102 includes a pair of chin support arm outer components 211a-b and a chin cup 212. The chin cup 212 is coupled on each end to inner ends of each of the pair of chin support arm outer components 211a-b. In an alternate embodiment the upper chin support arm 102 may be formed as a single component in place of the pair of chin support arm Outer components 211a-b and the chin cup 212. FIG. 4d shows a front view of the front assembly 200 having a single piece upper chin support arm 103. Similarly, FIG. 4e shows a rear view of the front assembly 200 having a single piece upper chin support arm 103.

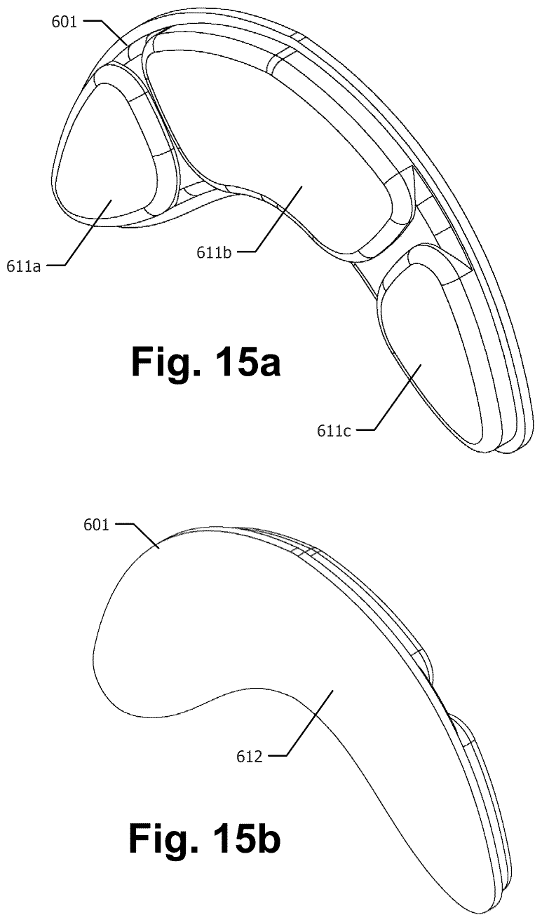

The combination of the chin cup 212 and the pair of chin support arm outer components 211a-b form a rigid upper chin support arm 102 that provides support to the user's head. The chin cup 212 is configured to create a resting position for the user's chin about the center of the upper chin support arm 102. The chin cup 212 may be covered by various materials as shown in FIGS. 15a-b to provide comfort to the user when the user's chin and jaw rests upon the chin cup 212. The chin cup 212 may also include padding under any covering material.

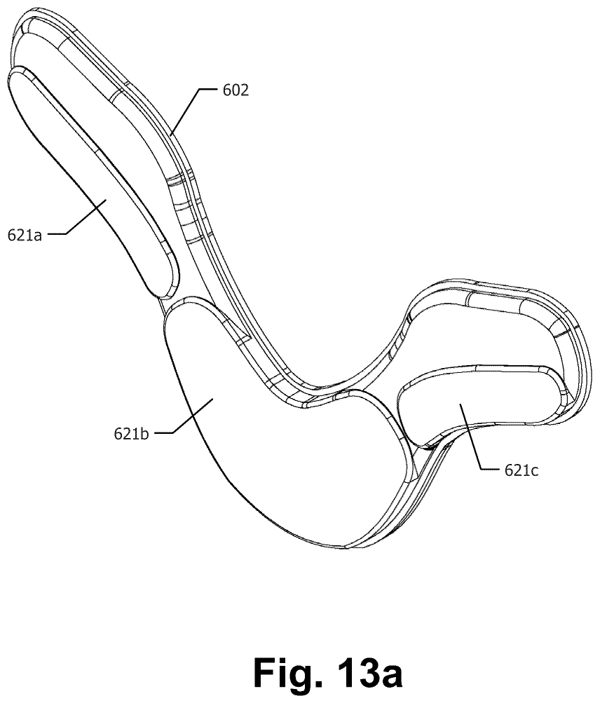

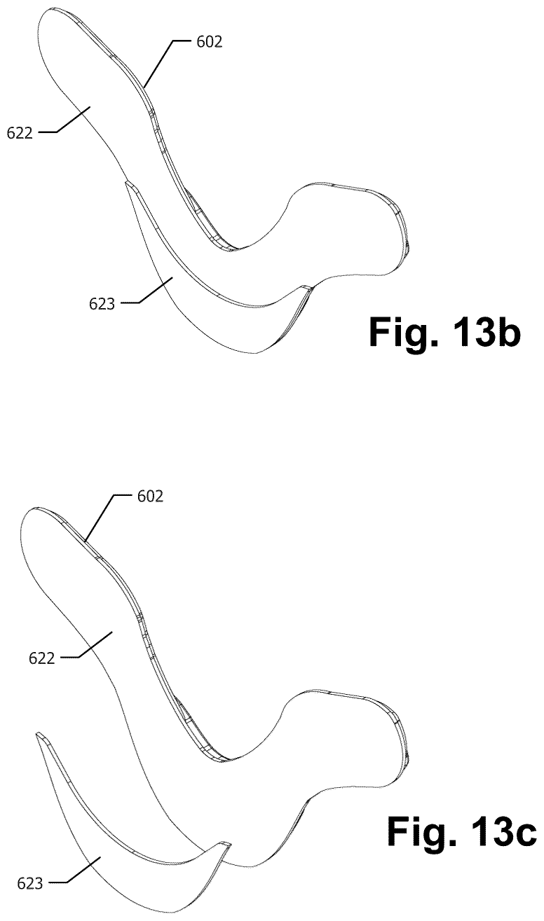

FIG. 4b shows a rear view of a front assembly 200 including the lower support arm 101 and upper chin support arm 102. The underside of the lower support arm 101 is the surface that is placed upon the user's chest when the cervical collar 100 is worn. The inner side of the lower support arm 101, in which the inner side of the lower support arm inner shell 202 is shown, is positioned about the user's neck when the cervical collar 100 is in place. Similar to the upper chin support arm 102 and the chin cup 212 described above, the lower support arm 101 may be covered by various padding materials as shown in FIGS. 13a-c to provide comfort to the user when the user's chest comes into contact with the lower support arm 101.

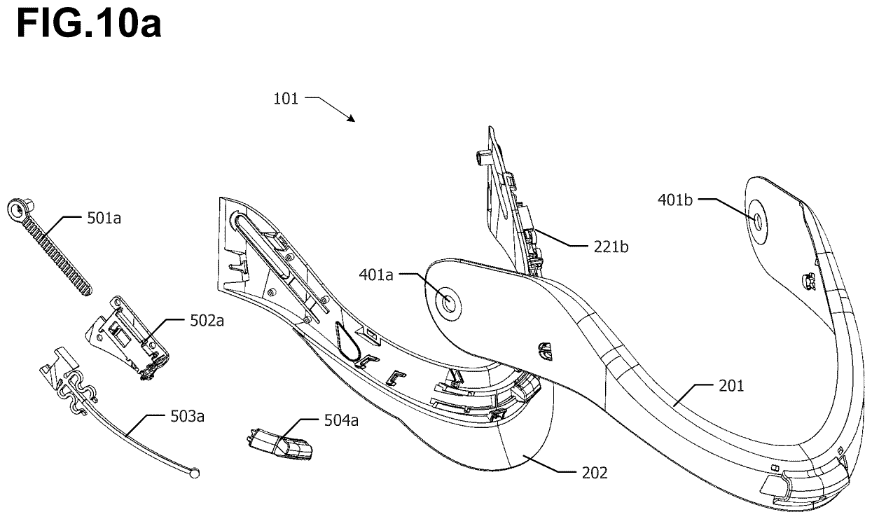

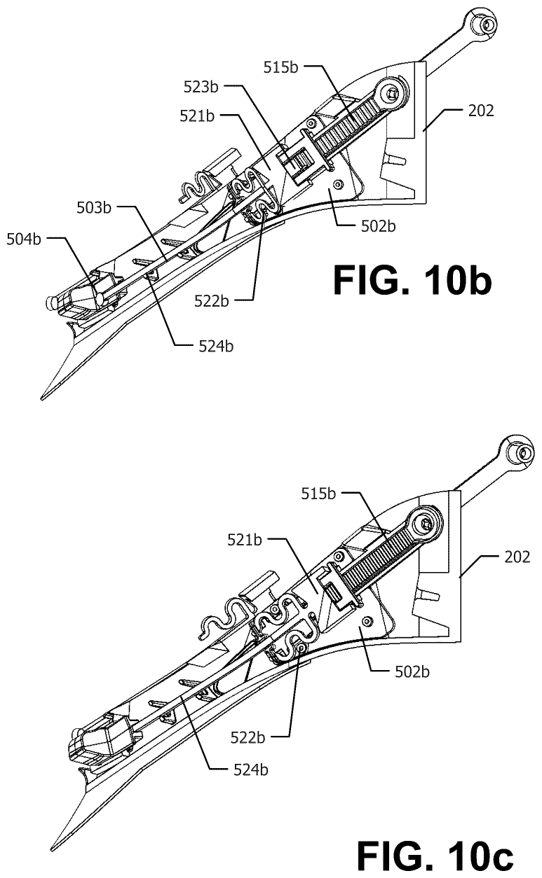

FIG. 4c shows an exploded rear view of a front assembly 200 including the lower support arm 101 and upper chin support arm 102. The components of the lower support arm locking mechanism 221a are shown adjacent to the right side of the front assembly 200. The chin cup 212 is shown in a center position of the upper chin support arm 102 between each of the chin support arm outer components 211a-b. The components of the lower support arm locking mechanism 221a include the toothed sliding height adjustment piece 501a having a plurality of teeth, the anchored locking tooth component 502a, the spring-loaded sliding lock 503a, and the locking mechanism release button 504a. The function and operation of these components within the lower support arm locking mechanism 221a-b is disclosed below in reference to FIGS. 10-12.

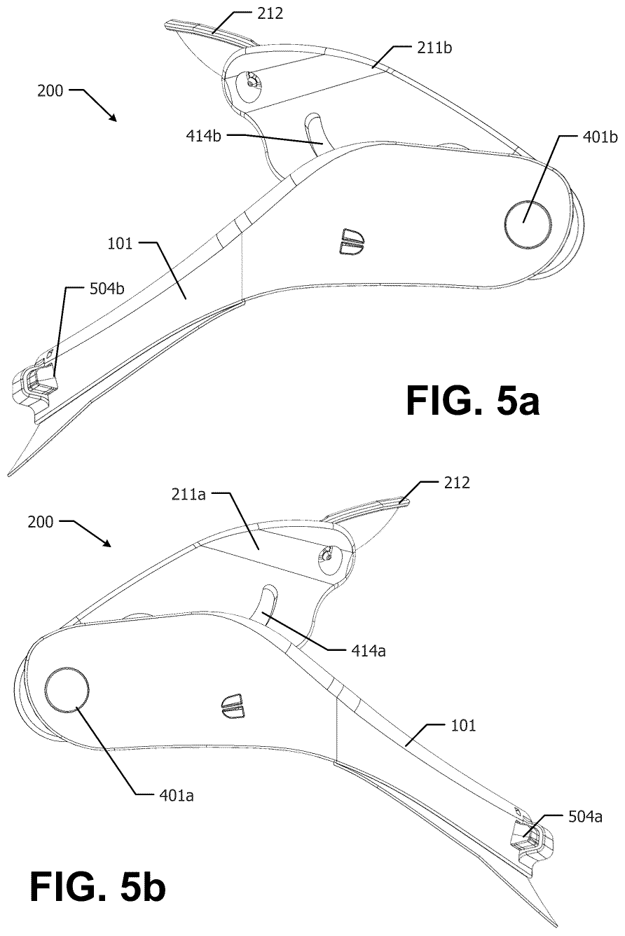

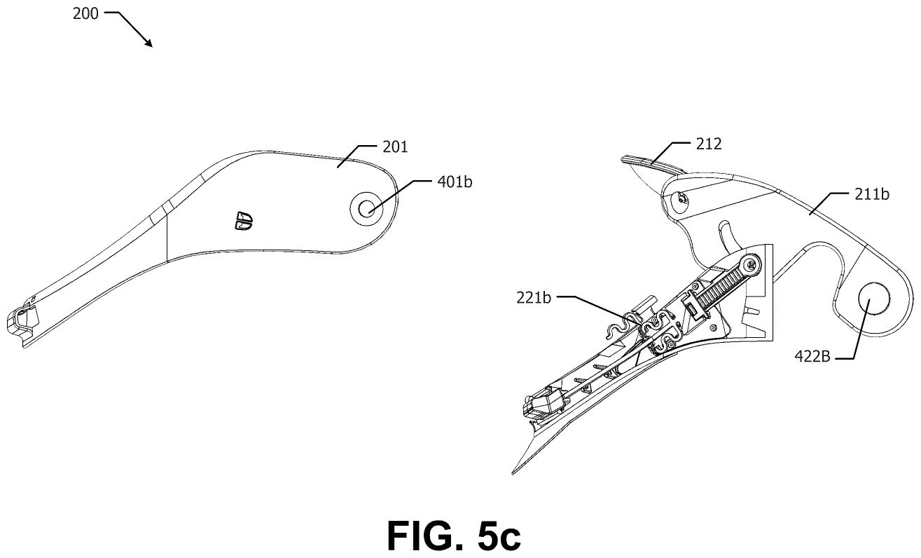

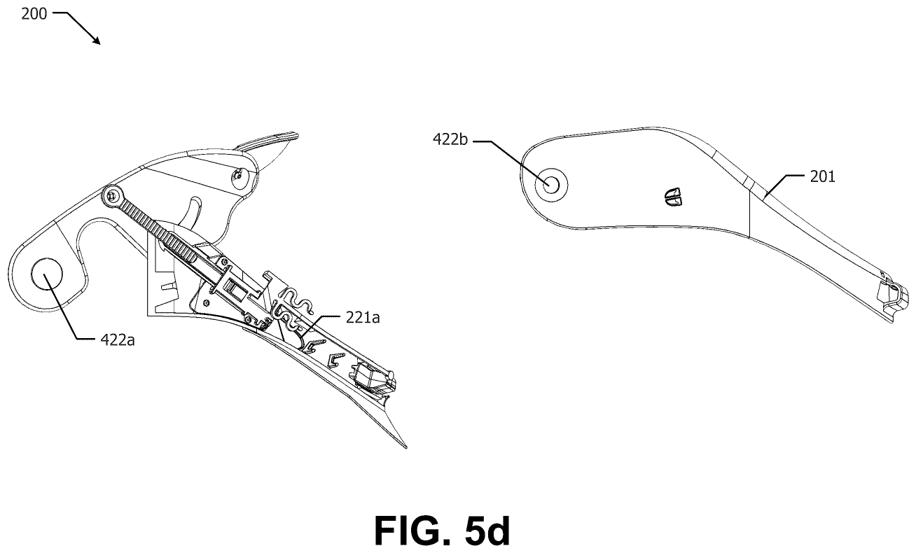

FIGS. 5a-d illustrate various side views of coupling a lower support arm and an upper chin support arm of an article of manufacture providing an adjustable cervical collar according to the present invention. FIG. 5a shows a right-side view of the lower support arm 101 pivotably coupled to the upper chin support arm 102. Similarly, FIG. 5b shows a left-side view of the lower support arm 101 pivotably coupled to the upper chin support arm 102. The lower support arm 101 has a lower support arm-upper chin support component attachment hole 401a-b through each end of the lower support arm 101, and chin support arm outer components 211a-b have upper support arm-lower support arm component attachment holes 422a-b that permit a coupling pin and receiver pair to be used to couple the lower support arm 101 and the upper chin support arm 102 together.

The lower support arm-upper chin support component attachment holes 401a-b and the upper support arm-lower support arm component attachment holes 422a-b create a pivot point about which the upper chin support arm 102 may rotate about the lower support arm 101. A pair of pivoting guide slots 414a-b is visible through the sides of the chin support arm outer components 211a-b. A pair of coupling pin and receiver pairs connect the chin support arm outer components 211a-b to the outer ends of the lower support arm 101, in which the pivoting guide slots 414a-b are configured to constrain the motion of the upper chin support arm 102 as it rotates about the pivot point between the lower support arm 101 and the upper chin support arm 102. Each of the pivoting guide slots 414a-b is curved slots through the sides of the chin support arm outer components 211a-b consistent with the rotation of the upper chin support arm 102 relative to the lower support arm 101.

The pair of locking mechanism release buttons 504a-b is visible at a center of the lower support arm 101 and connects to the lower support arm locking mechanisms 221a-b under the lower support arm outer shell 201 to lock and unlock the rotation of the upper chin support arm 102 from the lower support arm 101.

FIG. 5c shows a right-side exploded view of the lower support arm 101 pivotably coupled to the upper chin support arm 102. Similarly, FIG. 5d shows a left-side exploded view of the lower support arm 101 pivotably coupled to the upper chin support arm 102. In both FIGS. 5c and 5d, the lower support arm locking mechanism 221a-b is shown attached to the lower support arm inner shell 202. The lower support arm outer shell 201 is placed over the lower support arm locking mechanism 221a-b and the lower support arm inner shell 202 and is shown to encase the lower support arm locking mechanism 221a-b within the lower support arm 101. The upper support arm-lower support arm component attachment holes 422a-b and the lower support arm-upper chin support component attachment hole 401a-b are utilized when the front assembly 200 is formed by connecting the lower support arm 101 and the upper shin support arm 102.

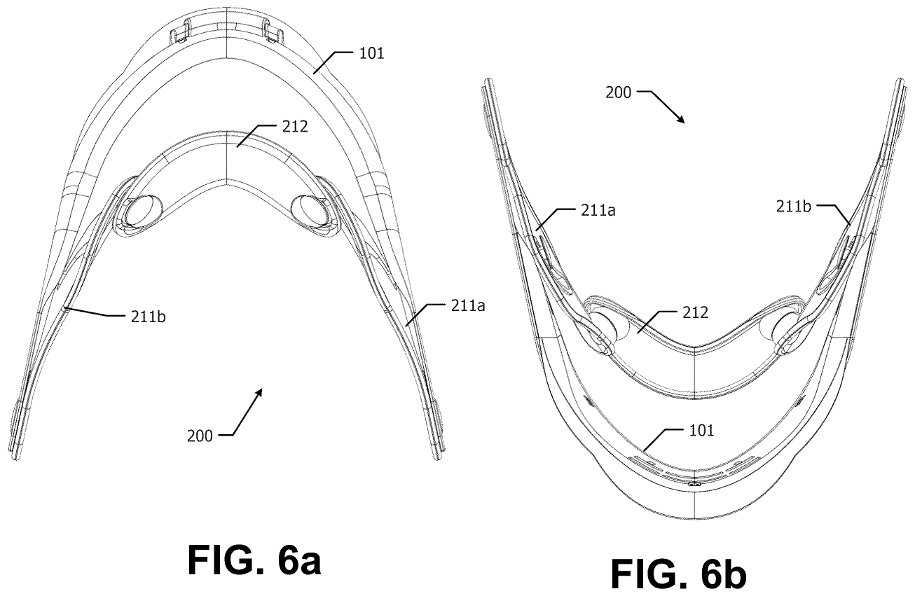

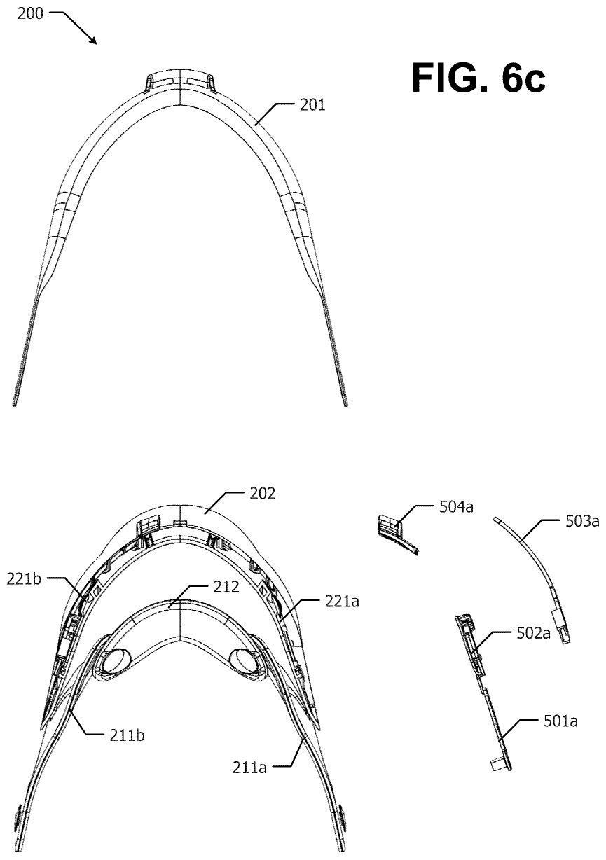

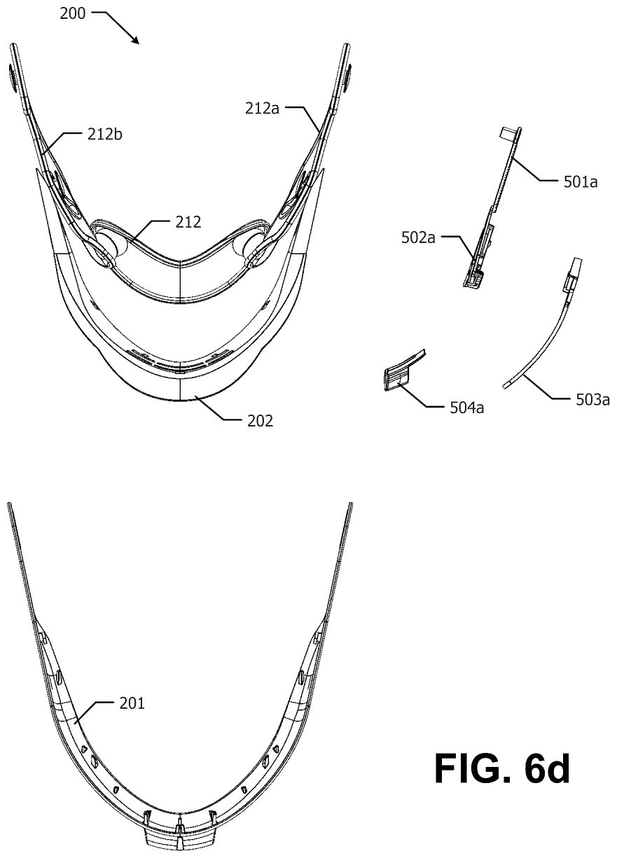

FIGS. 6a-d illustrate various top and bottom views of an article of manufacture providing an adjustable cervical collar according to the present invention. FIG. 6a shows a top view of the lower support arm 101 pivotably coupled to the upper chin support arm 102. FIG. 6b shows a bottom view of the lower support arm 101 pivotably coupled to the upper chin support arm 102. The chin cup 212 is visible within the center of the upper chin support arm 102 between the pair of chin support arm outer components 211a-b.

FIG. 6c shows an exploded top view of the lower support arm 101 pivotably coupled to the upper chin support arm 102. FIG. 6d shows an exploded bottom view of the lower support arm 101 pivotably coupled to the upper chin support arm 102. These exploded views of the front assembly 200 show the position of the pair of lower support arm locking mechanisms 221a-b. The pair of locking mechanism release buttons 504a-b is visible in the lower support arm outer shell 201 adjacent to each other within the center of the lower support arm outer shell 201.

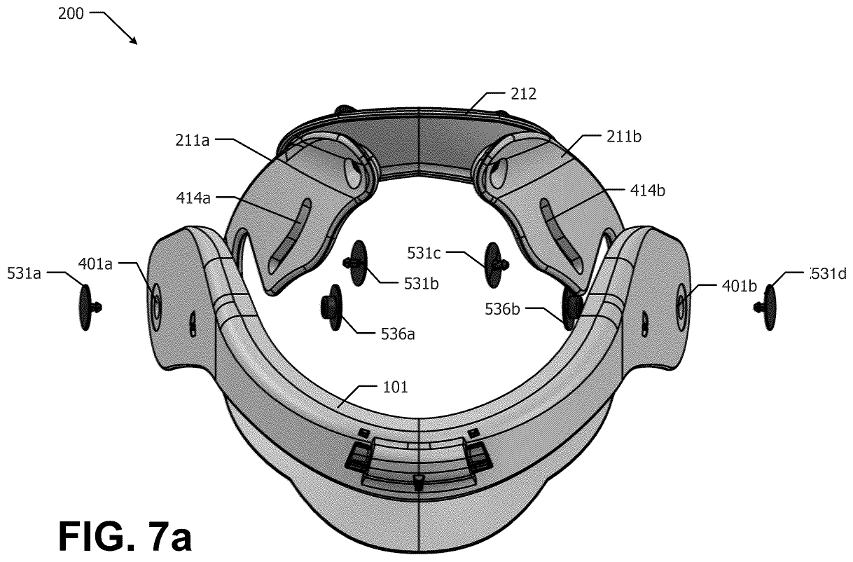

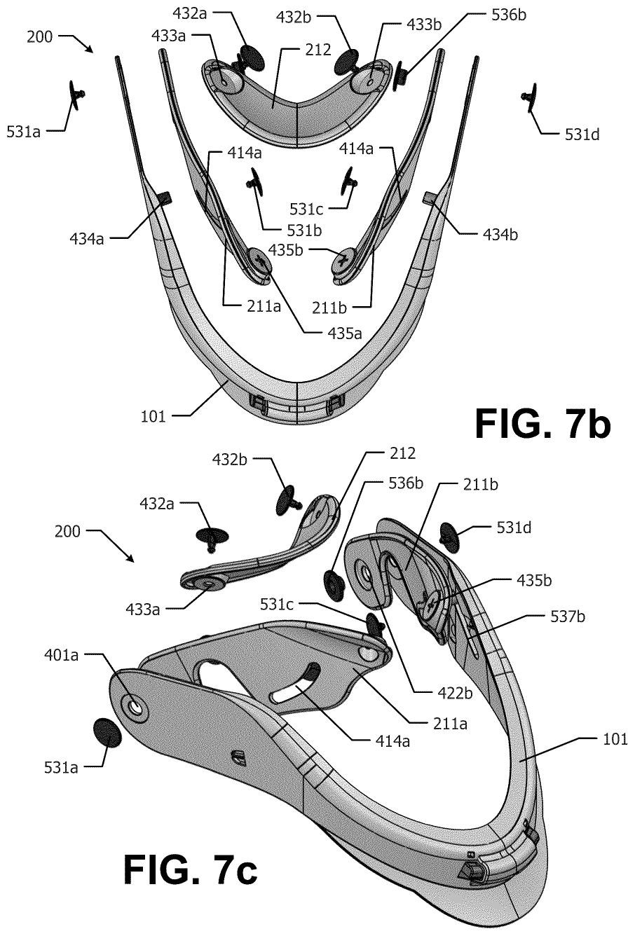

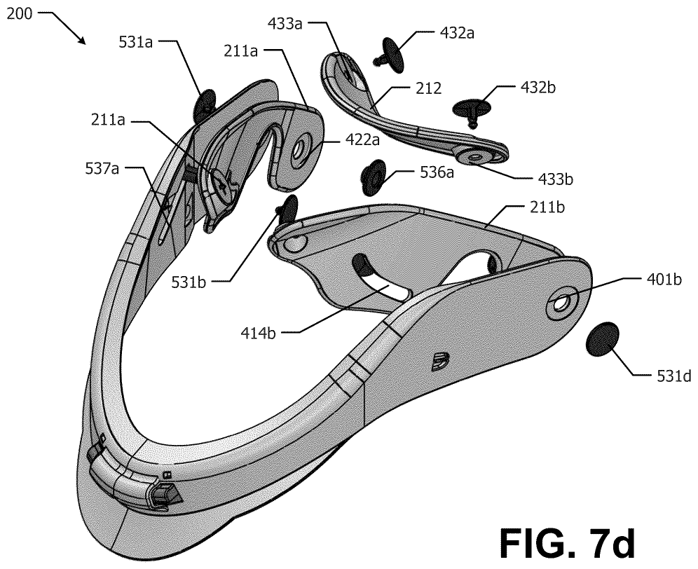

FIGS. 7a-d illustrate various views of coupling members between a lower support arm and an upper chin support arm of an article of manufacture providing an adjustable cervical collar according to the present invention. FIG. 7a shows an exploded front view of the lower support arm 101 pivotably coupled to the upper chin support arm 102 using the coupling attachment pins 531a-d and coupling attachment receivers 536a-b. The coupling attachment pins 531a, d attach to the mating coupling attachment receivers 536a-b after passing through the lower support arm-upper chin support component attachment hole 401a-b and upper support arm-lower support arm component attachment hole 422a-b. The coupling attachment pins 531a, d and coupling attachment receivers 536a-b engage each other to hold the lower support arm 101 and the chin support arm outer components 211a-b about the pivot points about the lower support arm-upper chin support component attachment holes 401a-b and upper support arm-lower support arm component attachment holes 422a-b.

The coupling attachment pins 531b, 531c couple the chin support arm outer components 211a-b to the lower support arm 101 as the coupling attachment pins 531b, c pass through the corresponding pivoting guide slots 414a-b in the chin support arm outer components 211a-b. The coupling attachment pins 531b, 531c engage within toothed sliding piece attachment pegs 434a-b as shown in figures below. The coupling attachment pins 531b, 531c create the rotational control through the pivoting guide slots 414a-b that constrain the rotation of the upper chin support arm 102 about the lower support arm 101. The toothed sliding piece attachment pegs 434a-b create height control by traveling through straight guide slots 537a-b and traverse through pivoting guide slots 414a-b. As toothed sliding piece attachment pegs 434a-b travel through both channels at the same time the angle changes between the upper chin support arm 102 and lower support arm 101.

FIG. 7b shows an exploded top view of the lower support arm 101 pivotably coupled to the upper chin support arm 102 using the toothed sliding piece attachment pegs 434a-b. In addition to the coupling attachment pins and receivers discussed above in reference to FIG. 7a, a pair of chin coupling attachment pins 432a-b and a corresponding pair of outer arm pin receivers 435a-b are shown that couple the chin support arm outer components 211a-b and the chin cup 212 together to form the upper chin support arm 102. The chin cup 212 is secured to each of the chin support arm outer components 211a-b by a pair of chin coupling attachment pins 432a-b that pass through corresponding chin cup holes 433a-b to connect to outer arm pin receivers 435a-b.

FIG. 7c shows an exploded left perspective view of the lower support arm 101 pivotably coupled to the upper chin support arm 102 using the toothed sliding piece attachment pegs 434a-b. FIG. 7d shows an exploded right perspective view of the lower support arm 101 pivotably coupled to the upper chin support arm 102. The two perspective views shown are mirror images of each other as the lower support arm 101 is coupled to the upper chin support arm 102 via the chin support arm outer components 211a-b and their corresponding attachment points.

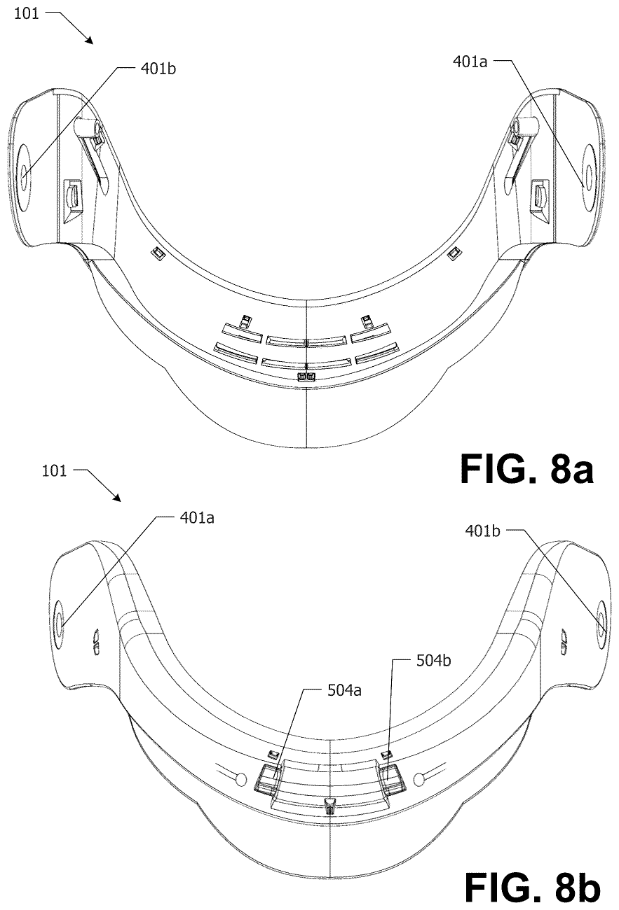

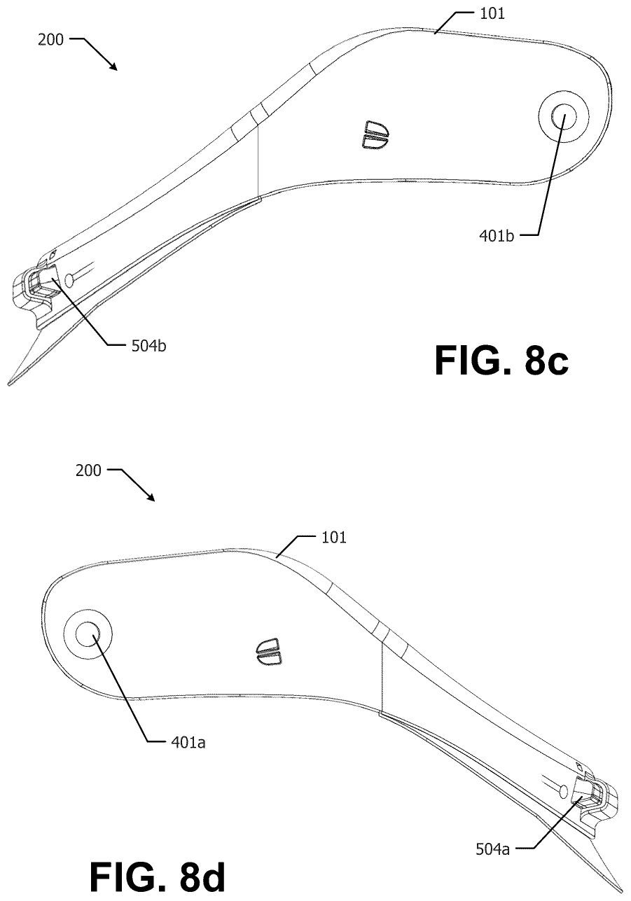

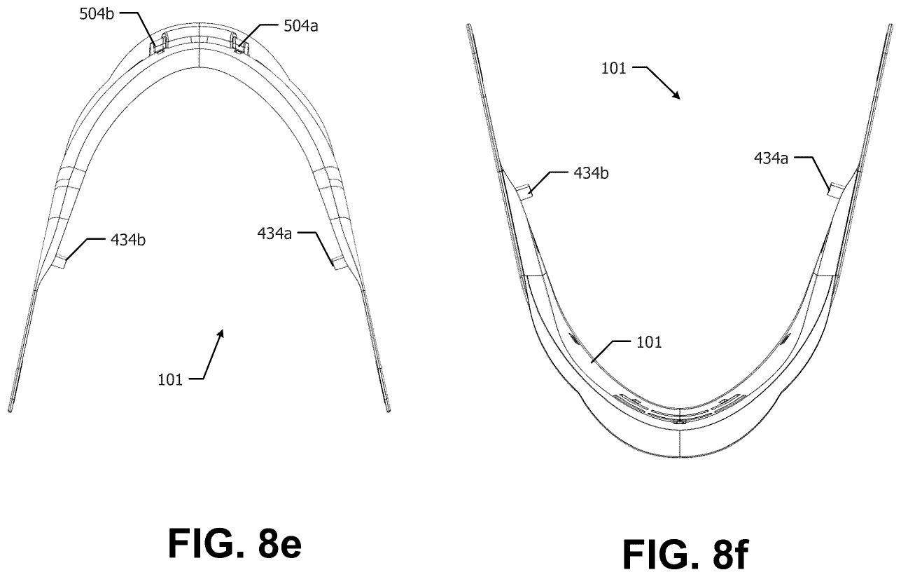

FIGS. 8a-f illustrate various views of a lower support arm of an article of manufacture providing an adjustable cervical collar according to the present invention. FIG. 8a shows a rear view of the lower support arm 101 and FIG. 8b shows a front view of the lower support arm 101. Similarly, FIG. 8c shows a right-side view of the lower support arm 101 and FIG. 8d shows a left side view of the lower support arm 101. Lastly, FIG. 8e shows a top view of the lower support arm 101 and FIG. 8f shows a bottom view of the lower support arm 101. In all of these views, the lower support arm-upper chin support component attachment holes 401a-b are shown about the outer ends of the lower support arm 101. The pair of locking mechanism release buttons 504a-b also are shown in views presenting a top side of the lower support arm 101.

FIGS. 9a-h illustrate various views of a rear support member of an article of manufacture providing an adjustable cervical collar according to the present invention. FIG. 9a shows a right perspective view of the rear support member 300 and FIG. 9b shows a left perspective view of the rear support member 300. Similarly, FIG. 9c shows a front view of the rear support member 300 and FIG. 9d shows a rear view of the rear support member 300. FIG. 9e shows a right-side view of the rear support member 300 and FIG. 9f shows a left side view of the rear support member 300. Lastly, FIG. 9g shows a top view of the rear support member 300 and FIG. 9h shows a bottom view of the rear support member 300.

In all of the above views of the rear support member 300, the rear support member 300 includes a rear support member frame component 301 and a pair of rear support member attachment loop straps 302a-b. As noted above with respect to FIGS. 3a-b, the rear support member attachment loop straps 302a-b are used to attach the rear support member frame component 301 to the front assembly 200 about a user's neck. Each of the rear support member attachment loop straps 302a-b loops through the corresponding rear support member attachment strap slots 315a-b and couples to itself. The rear support member attachment loop straps 302a-b may couple to itself after looping through the rear support member attachment strap slots 315a-b using hook and loop, adhesive, stitching, sonic welding, or similar attachment mechanism.

The rear support member frame component 301 includes a rear support member lower member 311, a pair of rear support member upper shoulder members 312a-b, and a pair of rear support member upper neck members 314a-b. The rear support member upper neck members 314a-b could be over-molded with more flexible material for more comfort. The rear support member lower member 311 is a single solid member extending downward from the center of the rear support member frame component 301, and could be an over-molded section. The rear support member lower member 311 is configured to be positioned along the upper spine of the user, typically between the shoulder blades.

Each of the rear support member upper shoulder members 312a-b extends outward from the center of the rear support member frame component 301. Both of the rear support member upper shoulder members 312a-b are configured to extend from the spine of the user both outward toward the ends of the front assembly 200 when placed about a user's neck and slightly upwards to configure the rear support member upper shoulder members 312a-b to rest across the top of the user's shoulders. Each of the rear support member upper shoulder members 312a-b is long enough to position the rear support member attachment loop straps 302a-b about the outer ends of the front assembly 200 and to hold the cervical collar 100 in place. The rear support member attachment loop straps 302a-b are configured to be attached to join the rear support member 300 to the front assembly 200 thereby creating the cervical collar 100. The rear support member attachment loop straps 302a-b may be repositioned along the adhesive hook on the front assembly 200 to adjust the tightness of the cervical collar 100 about the user's neck and head.

Each of the rear support member upper neck members 314a-b extends upward and slightly outward to create a support about a rear side of the user's neck. The rear support member upper neck members 314a-b curve inward to partially wrap around the sides of the user's neck allowing the rear support member frame component 301 to provide support to the user's head and neck while limiting the motion of the neck when the cervical collar 100 is in place.

Each of the rear support member attachment strap slots 315a-b is located within the corresponding rear support member upper shoulder members 312a-b and the rear support member attachment loop straps 302a-b are configured to loop through the rear support member attachment strap slots 315a-b and across the outer ends of the rear support member upper shoulder members 312a-b.

FIGS. 10a-c illustrate various views of a lower support arm having a pair of position locking mechanisms of an article of manufacture providing an adjustable cervical collar according to the present invention. FIG. 10a shows an exploded left perspective view of the lower support arm 101 showing the lower support arm locking mechanism 221a-b.

FIG. 10b shows a right-side view of the lower support arm locking mechanism 221a-b in an unlocked configuration. Similarly, FIG. 10c shows a right-side view of the lower support arm locking mechanism 221a-b in a locked configuration. Each of the lower support arm locking mechanisms 221a-b includes the toothed sliding height adjustment piece 501a-b, anchored locking tooth component 502a-b, the spring-loaded sliding lock 503a-b, and locking mechanism release buttons 504a-b as discussed above. The anchored locking tooth component 502a-b includes a latching tooth flexible arm portion 523a-b that engages the toothed sliding height adjustment piece 501a-b to lock the chin support arm outer components 211a-b. An upper end of each of the latching tooth flexible arm portion 523a-b is coupled to the corresponding toothed sliding height adjustment piece 501a-b, while the upper end of each of the toothed sliding height adjustment piece 501a-b is secured against the lower support arm inner shell 202 by the anchored locking tooth component 502a-b and can slide upward and downward within the anchored locking tooth component 502a-b.

Each of the spring-loaded sliding locks 503a-b has a corresponding release button coupling elongated section 524a-b release that connects the corresponding spring-loaded sliding lock 503a-b to the locking mechanism release buttons 504a-b. The locking mechanism release buttons 504a-b extend outward through the lower support arm outer shell 201 permitting the user to depress the locking mechanism release buttons 504a-b to unlock the lower support arm locking mechanism 221a-b.

When a user depresses one or both locking mechanism release buttons 504a-b, the locking mechanism release buttons 504a-b move toward the center of the lower support arm 101. The spring-loaded sliding lock 503a-b is pulled downward as the locking mechanism release button 504a-b is depressed causing the spring-loaded sliding lock 503a-b to slide downward. A spring engagement mechanism 522a-b is coupled to a top end of the spring-loaded sliding lock 503a-b and is configured to be positioned within the anchored locking tooth component 502a-b to disengage the tooth engagement body 521a-b from the latching tooth flexible arm portion 523a-b. When the tooth engagement body 521a-b slides downward, it disengages the latching tooth flexible arm portion 523a-b permitting the latching tooth flexible arm portion 523a-b to freely lift off of the toothed sliding height adjustment piece 501a-b to cause the chin support arm outer component 211a-b to unlock. When the lower support arm locking mechanism 221a-b is unlocked, the toothed sliding height adjustment piece 501a-b may slide up and down within the lower support arm locking mechanism 221a-b as the upper chin support arm 102 rotates with respect to the lower support arm 101.

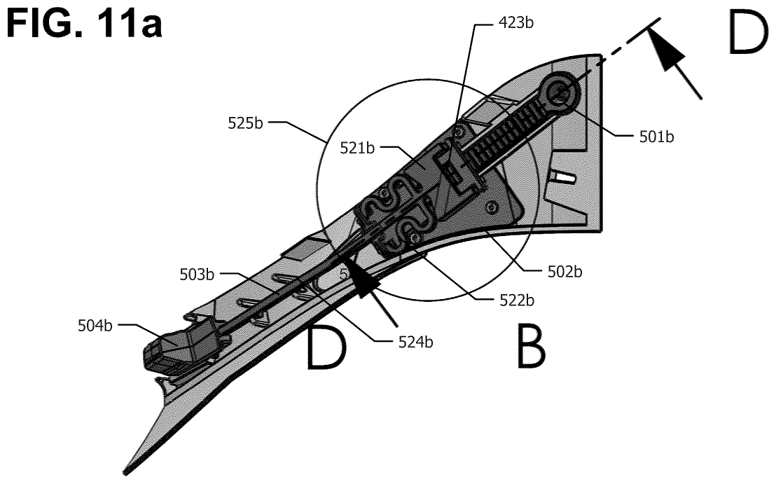

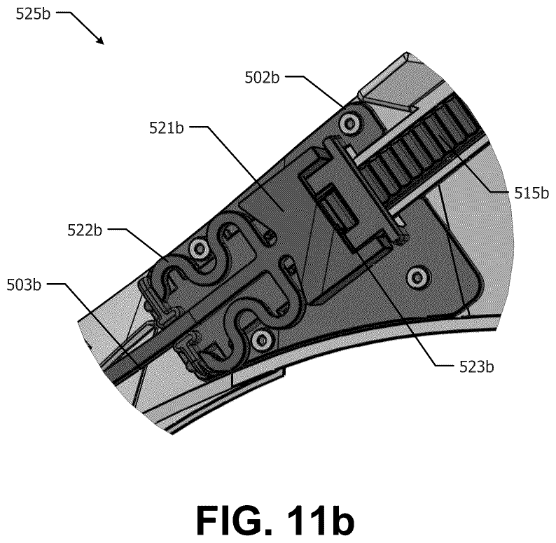

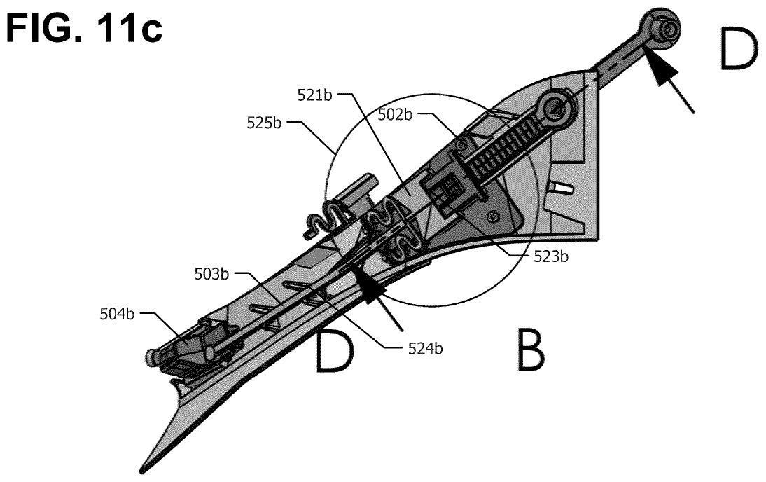

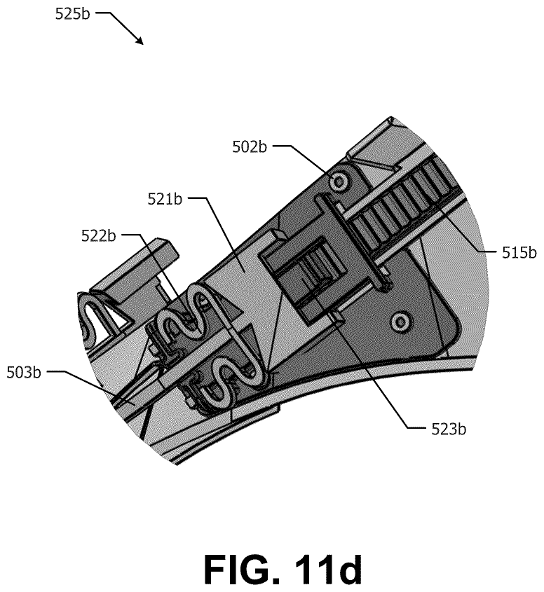

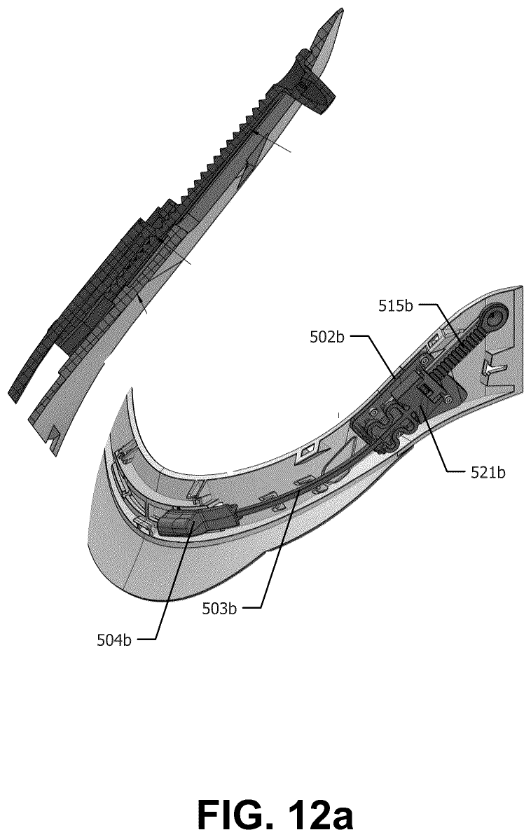

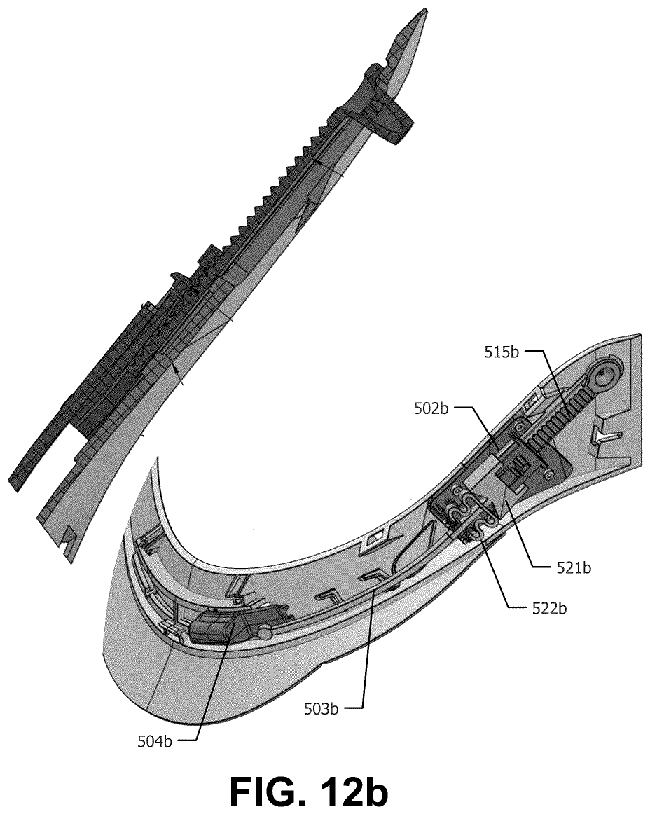

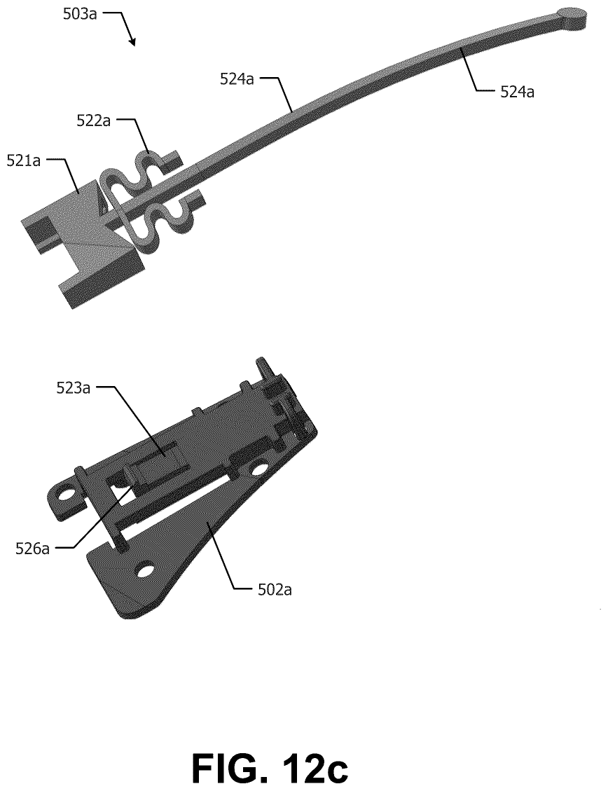

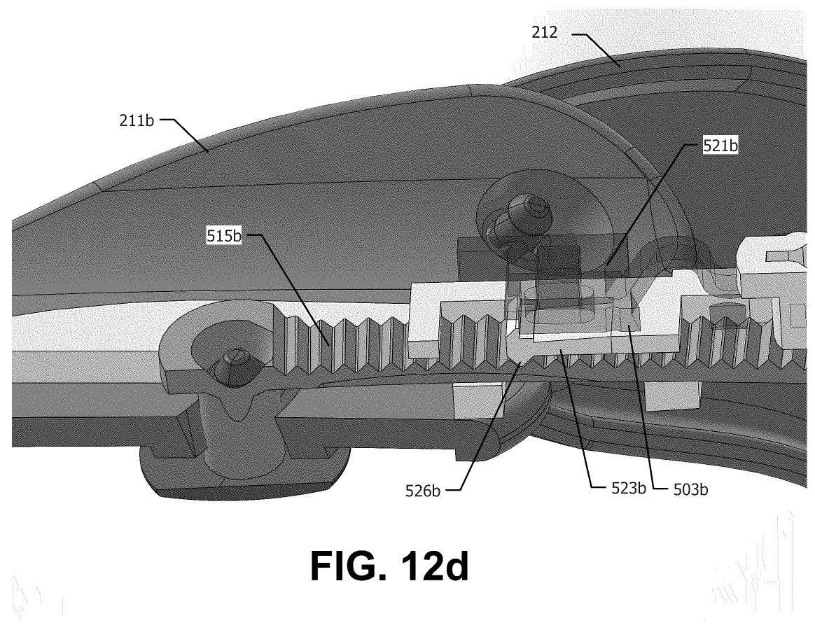

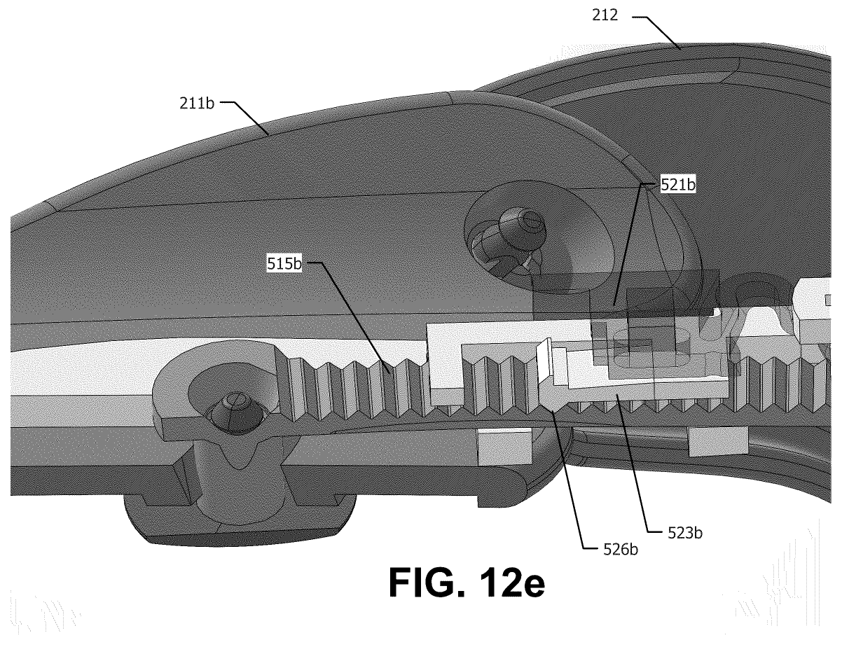

When the user releases the locking mechanism release buttons 504a-b, the spring-loaded sliding lock 503a-b is pulled upward by the spring engagement mechanism 522a-b allowing the tooth engagement body 521a-b to slide to reengage the latching tooth flexible arm portion 523a-b forcing the arm downward to reengage the toothed sliding height adjustment piece 501a-b to relock the lower support arm locking mechanism 221a-b. The operation of the latching mechanism in the lower support arm locking mechanism 221a-b is disclosed in detail below in reference to FIGS. 11a-f and FIGS. 12a-e.

FIGS. 11a-d illustrate various views of components of each of the pair of position locking mechanisms of an article of manufacture providing an adjustable cervical collar according to the present invention. FIG. 11a shows a right side view of the lower support arm locking mechanism 221a-b in a locked position. A corresponding detailed right side view 525b is shown in FIG. 11b of the lower support arm locking mechanism 221a in a locked position. Similarly, FIG. 11c shows a right side view of the lower support arm locking mechanism 221a in an unlocked position. A corresponding detailed right side view 525b is shown in FIG. 11d of the lower support arm locking mechanism 221a in an unlocked position.

In FIGS. 11a-b, the spring engagement mechanism 522a-b are shown in an uncompressed configuration which creates interference between the spring engagement mechanism 522a-b and the anchored locking tooth component 502a-b. The interference between the spring engagement mechanism 522a-b and the anchored locking tooth component 502a-b prevents the tooth engagement body 521a-b from disengaging with the latching tooth flexible arm portion 523a-b, pushing the arm downward to engage the teeth in the toothed sliding height adjustment piece 501a-b, preventing the toothed sliding height adjustment piece 501a-b from sliding upward or downward within the anchored locking tooth component 502a-b. When the toothed sliding height adjustment piece 501a-b is not constrained, it may slide upward and downward within straight guide slot 537a-b to permit the upper chin support arm 102 to rotate about the lower support arm 101.

FIGS. 12a-d illustrate open and closed configurations of each of the pair of position locking mechanisms of an article of manufacture providing an adjustable cervical collar according to the present invention. FIG. 12a shows a left side view of the locking mechanism latching teeth 515a-b that engage the lower support arm locking mechanism 221a-b when the locking mechanism is configured in a locked position. Similarly, FIG. 12b shows a left side view of the locking mechanism latching teeth 515a-b that engages the lower support arm locking mechanism 221a-b when the locking mechanism is configured in an unlocked position. The operation of the lower support arm locking mechanism 221a-b in these figures is described in detail above with reference to FIGS. 10-11.

FIG. 12c shows components of anchored locking tooth component 502a-b and spring-loaded sliding lock 503a-b. The spring-loaded sliding lock 503a-b includes the release button coupling elongated section 524a-b, a spring engagement mechanism 522a-b, and a tooth engagement body 521a-b. The anchored locking tooth component 502a-b includes the latching tooth flexible arm portion 523a-b coupled at an inner end of the arm to a top surface of the anchored locking tooth component 502a-b. Each of the latching tooth flexible arm portion 523a-b further includes an engagement tooth 526a-b at an outer end of the latching tooth flexible arm portion 523a-b that engages the locking mechanism latching teeth 515a-b of the toothed sliding height adjustment piece 501a-b. The spring-loaded sliding lock 503a-b slides across the top surface of anchored locking tooth component 502a-b when the spring engagement mechanism 522a-b are uncompressed as shown in FIG. 12d. When the spring engagement mechanism 522a-b are pulled into a compressed configuration when the locking mechanism release buttons 504a-b are depressed by the user, the spring-loaded sliding lock 503a-b slide in the direction of the locking mechanism release buttons 504a-b to disengage the latching tooth flexible arm portion 523a-b allowing the arm to rise free from the locking mechanism latching teeth 515a-b of the toothed sliding height adjustment piece 501a-b as shown in FIG. 12e.

FIGS. 13a-c illustrate multiple views of a lower support arm padding 602 used with the lower support arm 101 of an article of manufacture providing an adjustable cervical collar according to the present invention. FIG. 13a shows a front perspective view of the lower support arm padding 602 having a plurality of raised padding sections 621a-c. FIG. 13b shows a rear perspective view of the lower support arm padding 602 showing a rear surface 622 and a lower pocket outer item 623 coupled to the rear surface 622 of the lower support arm padding 602. FIG. 13c shows an exploded rear perspective view of the lower support arm padding 602 showing the lower pocket outer item 623. The lower pocket outer item 623 may be utilized to wrap around corresponding components of the lower support arm 101 to hold the lower support arm padding 602 in place when the adjustable cervical collar 100 is in use.

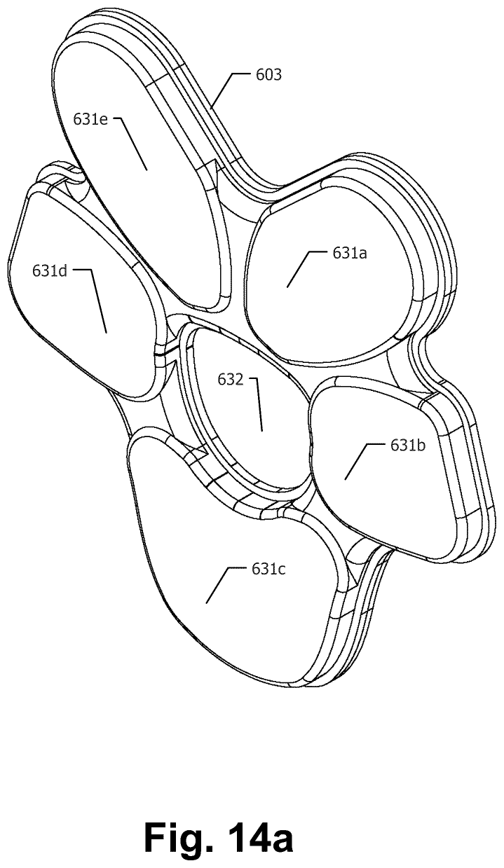

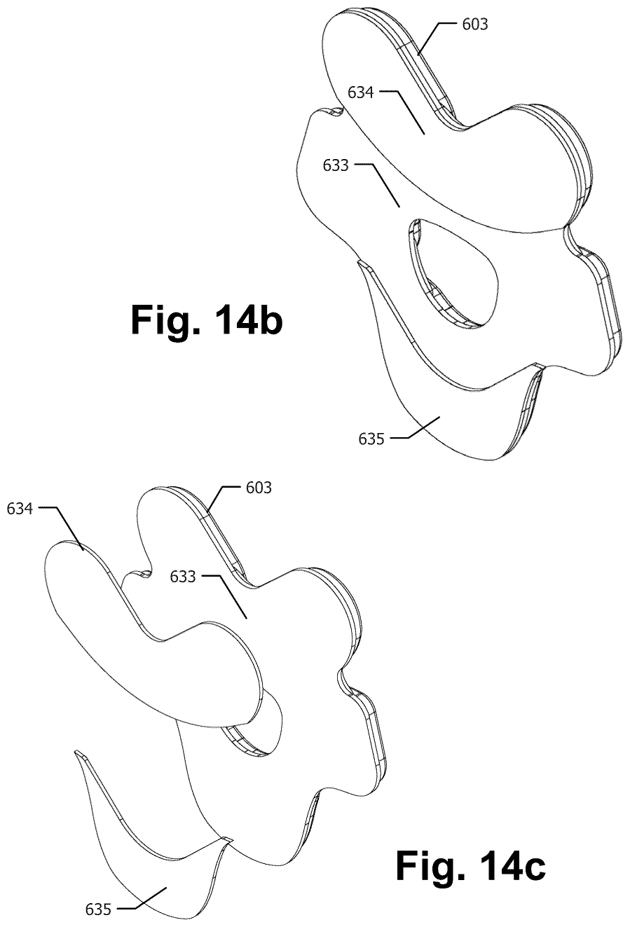

FIGS. 14a-c illustrate multiple views of a rear support member padding 603 used with the rear support member 300 of an article of manufacture providing an adjustable cervical collar according to the present invention. FIG. 14a shows a front perspective view of the rear support member padding 603 having a plurality of raised padding sections 631a-e and a center hole 632. FIG. 14b shows a rear perspective view of the rear support member padding 603 showing a rear surface 633, a top pocket outer item 634, and a lower pocket outer item 635 coupled to a rear surface 633 of the rear support member padding 603. FIG. 14c shows an exploded rear perspective view of the rear support member padding 603 showing the rear surface 633, the top pocket outer item 634, and a lower pocket outer item 635. The top pocket outer item 634 and the lower pocket outer item 635 may be utilized to wrap around corresponding components of the rear support member 300 to hold the rear support member padding 603 in place when the adjustable cervical collar 100 is in use.

FIGS. 15a-b illustrate multiple views of an upper chin support arm padding 601 used with the upper chin support arm 102 of an article of manufacture providing an adjustable cervical collar according to the present invention. FIG. 15a shows a front perspective view of the upper chin support arm padding 602 having a plurality of raised padded sections 611a-c. FIG. 15b shows a rear perspective view of upper chin support arm padding 601 having a rear surface 612. Attachment mechanisms including hook and loop devices, adhesive, and similar devices may be utilized to hold the upper chin support arm padding 601 in place when the adjustable cervical collar 100 is in use.

With respect to the padding items including the lower support arm padding 602, upper chin support arm padding 601, and the rear support member padding 603, the padding may be constructed from padding material over-molded into a desired shape, cut and shaved into a desired shape, or shaped into a desired configuration using similar construction methods. The padding items 601-603, each item may be covered using fabric or similar materials that are laminated, sewn, or otherwise attached to the padding to provide a desired outer surface for contacting the user when the adjustable cervical collar 100 is worn.

Even though particular combinations of features are recited in the present application, these combinations are not intended to limit the disclosure of the invention. In fact, many of these features may be combined in ways not specifically recited in this application. In other words, any of the features mentioned in this application may be included in this new invention in any combination or combinations to allow the functionality required for the desired operations.

No element, act, or instruction used in the present application should be construed as critical or essential to the invention unless explicitly described as such. Further, the phrase “based on” is intended to mean “based, at least in part, on” unless explicitly stated otherwise.

What is claimed is:

1. An article of manufacture for providing an adjustable cervical collar, the adjustable cervical collar comprising:
a lower support arm having a pair of opposing ends;
a rear support member attachable from the lower support arm;
a pair of lower support arm locking mechanisms coupled to the lower support arm and having a locked and unlocked configuration;
a pivotable upper chin support arm having opposing ends and coupled to the lower support arm at the opposing ends;
wherein the lower support arm and the upper chin support arm rotate about a connecting pivot point when the pair of lower support arm locking mechanisms are in an unlocked position; and
the pair of lower support arm locking mechanisms constrains the pivoting positioning of the lower support arm relative to the pivotable upper chin support arm in the locked configuration.

2. The adjustable cervical collar according to claim 1, wherein each of the pair of lower support arm locking mechanisms comprises:
a toothed sliding height adjustment piece having a plurality of teeth;
an anchored locking tooth component;
a spring-loaded sliding lock;
a locking mechanism release button coupled to the release button coupling elongated section.

3. The adjustable cervical collar according to claim 2, wherein the anchored locking tooth component comprises:
a top surface;
a latching tooth flexible arm portion having an inner end being coupled to the top surface; and
an engagement tooth at an outer end of the latching tooth flexible arm portion;

4. The adjustable cervical collar according to claim 2, wherein the spring-loaded sliding lock comprises:
a tooth engagement body;
spring engagement mechanism; and
a release button coupling elongated section.

5. The adjustable cervical collar according to claim 3, wherein the toothed sliding height adjustment piece comprises a locking mechanism latching teeth for engaging a latching tooth engagement arm of the anchored locking tooth component.

6. The adjustable cervical collar according to claim 5, wherein depressing the locking mechanism release button pulls the release button coupling elongated section causing the spring engagement mechanism to compress and the tooth engagement body to disengage from the latching tooth engagement arm to permit the engagement tooth to disengage the locking mechanism latching teeth.

7. The adjustable cervical collar according to claim 6, wherein releasing the locking mechanism release button causes the spring engagement mechanism to return to an uncompressed state pushing the tooth engagement body to engage the latching tooth flexible arm portion causing the engagement tooth to reengage the locking mechanism latching teeth.

8. The adjustable cervical collar according to claim 2, wherein the lower support arm comprises:
a lower support arm outer shell; and
a lower support arm inner shell, the pair of lower support arm locking mechanisms being coupled to the lower support arm inner shell.

9. The adjustable cervical collar according to claim 8, wherein the locking mechanism release button is accessible from outside the lower support arm outer shell.

10. The adjustable cervical collar according to claim 2, wherein a rear support member attachable from the lower support arm, the rear support member comprising:
a rear support member frame component and
a pair of rear support member attachment straps for coupling the rear support member to the lower support arm.

11. The adjustable cervical collar according to claim 10, wherein the rear support member frame component comprises:
a pair of rear support member attachment strap slots for accepting corresponding rear support member attachment straps;
a rear support member lower member;
a pair of rear support member upper shoulder members; and
a pair of rear support member upper neck members.

12. The adjustable cervical collar according to claim 1, further comprises:
a lower support arm padding coupled to the lower support arm using one or more first lower pocket outer item;
a rear support member padding coupled to the rear support member using a top pocket outer item and a second lower pocket outer item; and
an upper chin support member padding coupled to the upper chin support arm.

13. The adjustable cervical collar according to claim 12, wherein the upper chin support arm padding, the lower support arm padding, and the rear support member padding each having a laminated outer layer and a plurality of plurality of raised padding sections.

14. The adjustable cervical collar according to claim 13, wherein the plurality of raised padding sections being molded into a single layer.
